(19) United States
(12) Patent Application Publication
(10) Pub. No.: US 2001/0051748 A1
(43) Pub. Date: Dec. 13, 2001

(54) Polymerizable compounds, and their use as wax

(76) Inventors: Franz-Leo Heinrichs, Gablingen (DE); Ernst Krendlinger, Friedberg (DE); Heinz Ehrhardt, Rehling (DE); Andree Henze, Augsburg (DE)

Correspondence Address: Clariant Corporation, Industrial Property Department, 4331 Chesapeake Drive, Charlotte, NC, 28216, US

(21) Appl. No.: 09/770,917

(22) Filed: Jan. 26, 2001

(30) Foreign Application Priority Data
Jan. 26, 2000 (DE) 10003118.8

(51) Int. Cl. C07C 69/52; C07C 57/13

(52) U.S. Cl. 562/595; 560/190

(57) ABSTRACT

The invention relates to ethylenically unsaturated compounds of the formula

Zₖ—(Y—(W)₍ₘ₋ₚ₋₁₋ₖ₎(X)_{q})ₙ

and their use as copolymerizable wax constituent.

### BACKGROUND OF THE INVENTION

The present invention relates to ethylenically unsaturated compounds having long alkyl chains, and to their use as copolymerizable wax constituent for surface coatings.

Areas of application for wax-like substances are, for example, the areas of hydrophobicization, matting, dispersion, release, plasticization, viscosity regulation, coating, smoothing of surface structures, emulsification, melting point adjustment, lubrication, retardation, gloss adjustment, flexibilization, surface protection, binders and compatibilization.

In most applications, the wax is employed as additive and remains in the end product. Good compatibility and high molecular weights support this remaining in a substrate matrix, but increasingly substrates/products, for example, in contact with food, are required to release no constituents to the environment, neither through exudation nor by extraction. This applies both to the base materials and to the additives, such as, for example, waxes.

In the area of UV-curing coatings, in particular in paints and printing inks, the proportion of extractable substances, principally monomers and reactive thinners, has been reduced by optimizing the curing process.

As additives for improving viscosity, flow behavior and tack, DE-A-24 23 354 describes partial esters of polyhydric alcohols which contain a plurality of acrylic ester groups and can thus be copolymerized into the film in a network-like manner. These are products which as intended are highly compatible with the binder system. However, they have excessively low melting points.

In additives for the adjustment of abrasion protection, sliding friction, surface grip, gloss and matting, such as, for example, micronized waxes, which are in the form of particles in the film, the problem of durable binding of the particles into the film has still not been solved. The wax can be eluted both from the surface and from the film by extraction.

### SUMMARY OF THE INVENTION

On the basis of this problem, the object of the present invention was to find waxes which are distinguished in the surface coating by increased retention in the coating material. In addition, the waxes should have adequate mechanical strength for surface coatings.

Surprisingly, it has been found that wax-like compounds, which can be bound in reactively, achieve this object if they have limited proportions of polymerizable double bonds.

### DESCRIPTION OF THE PREFERRED EMBODIMENTS

The invention therefore relates to compounds of the formula

Zₖ—(Y—(W)₍ₘ₋ₚ₋₁₋ₖ₎(X)_{q})ₙ

in which

Z is the radical obtained by removing the OH groups from COOH groups of an organic carboxylic acid containing 1-4 COOH groups and whose number of carbon atoms is between 4 and 40,

Y is the radical obtained by removing (m-p) hydrogen atoms from the hydroxyl groups of an organic compound having 3 to 15 carbon atoms and containing m OH groups, where Y can be a radical alkoxylated by C₂-C₄-alkylene oxides, and of the total number m of OH groups, (m-p) OH groups have been esterified and p OH groups are in free or etherified form,

X is the monovalent radical obtained by removing the OH group from the COOH group of an organic monocarboxylic acid having 3-5 carbon atoms which contains an olefinic double bond which is in conjugation with the C—O double bond,

W is the monovalent radical obtained by removing the OH group from the COOH group of a monobasic carboxylic acid having 16-60 carbon atoms,

p is a number from 0 to 2.5 which indicates the number of free or etherified OH groups,

k is a number from 0 to 2,

q is a number from 0.5 to 1.5 which indicates the number of ester units in the unsaturated acid X,

n is an integer from 1 to 6, with the proviso that k is >0, in which case n indicates the number of recurring units in the compound according to the invention,

m is an integer from 3 to 8 and indicates the number of OH groups in the polyhydric alcohol,

with the proviso that

1≦n.q≦6,

2≦(n[m-p-1])≦15,

m-p-1>0, and

m-n-1-k>1.

The monocarboxylic acid described under W must have a hydrophobic character. It is therefore necessary to use C₁₆-C₆₀-, preferably C₂₂-C₄₀-carboxylic acids, as the pure compound or as an acid mixture. For the desired product properties, it is important that the main fraction of the acid comprises saturated, linear hydrocarbon chains. Unsaturated and branched carboxylic acids may also be present in a mixture of this type to a secondary extent of up to 10 mol %. Examples of suitable compounds are tallow fatty acid, montan wax acid, paraffin oxidates, wax oxidates, olefin oxidates, technical-grade C₃₆ Guerbet acid, behenic acid and erucic acid.

In order to achieve the desired molecular weight and polarity, the polybasic carboxylic acids described under Z can be used if desired, for example, hydrogenated dimeric fatty acids, hydrogenated trimeric fatty acids, dodecanedioc acid, adipic acid, alkylsuccinic acid or alkenylsuccinic acid.

The polyhydric alcohols described under Y are at least trihydric alcohols and/or their ethoxylation products, such as, for example, glycerol, trimethylolpropane, pentaerythritol and sorbitol. Also suitable are the dimers and trimers of said alcohols, such as, for example, diglycerol, so long as they do not contain more than 8 OH groups and do not have more than 15 carbon atoms. If Y is alkoxylated, it carries from 0 to 20 alkoxy groups, preferably ethoxy or propoxy groups. The number of 3 to 15 carbon atoms which the polyhydric alcohol Y contains applies exclusively to any alkoxy groups which may be present.

The radical of an ethylenically unsaturated acid denoted by X in the formula preferably originates from acrylic acid, methacrylic acid or esters thereof.

The total number of ester units in the ethylenically unsaturated acid X, n.q, is preferably between 1 and 4.

The compounds according to the invention are generally prepared in a two-step process.

In the first step, the pre-ester is prepared from the polyhydric alcohol Y, the long-chain carboxylic acid W and, if desired, a dicarboxylic acid Z. The reaction is carried out without solvent using standard esterification catalysts, such as methanesulfonic acid, acidic ion exchangers, Sn compounds, titanic acid esters or Na methoxide. After the requisite acid number (AN) has been set, the ethylenically unsaturated acid X is then, in a second step, without work-up, incorporated by direct esterification or transesterification. If desired, the catalyst can be changed for the second step. The stoichiometry for the incorporation of the unsaturated acid is selected in such a way that at least 1 mol, but at most 6 mol, of unsaturated acid is incorporated. Higher proportions of double bonds reduce the hardness of the product.

The compounds according to the invention are wax-like compounds which are solid at room temperature and can be employed as solids in micronized form, melts or dispersion form like known waxes.

Wax is a technological generic term for a series of natural or synthetically prepared substances which generally have the following properties: plastic at 20° C., solid to brittle and hard, coarse to finely crystalline, translucent to opaque, not glass-like, melting at above 40° C. without decomposition, relatively low viscosity at slightly above the melting point, highly temperature-dependent consistency and solubility, polishable under gentle pressure.

### EXAMPLES

### Example 1

| | | |
|---|---|---|
| Trimethylolpropane | 1.0 | mol |
| Montanic acid | 1.5 | mol (C₂₈-carboxylic acid) |
| Acrylic acid | 1.1 | mol |
| Methanesulfonic acid, 70% | 0.15% | by weight |

The montanic acid is melted, and trimethylolpropane and methanesulfonic acid are added. The mixture is then esterified at 120° C. until an AN of <10 has been reached. The acrylic acid is added and esterified until an AN of <10 has been reached. The catalyst is neutralized using aqueous alkali, and residues of volatile constituents are removed under reduced pressure. The batch is filtered and compounded. The resultant product exhibits the following properties:

| | |
|---|---|
| AN | 8.5 |
| SN | 61 |
| DP | 78° C. |
| MV 90 | 70 mPas |

### Example 2

| | | |
|---|---|---|
| Trimethylolpropane | 1.0 | mol |
| Montanic acid | 1.1 | mol |
| Adipic acid | 0.2 | mol |
| Acrylic acid | 1.1 | mol |
| Methanesulfonic acid, 70% | 0.15% | by weight |

The montanic acid is melted, and trimethylolpropane and methanesulfonic acid are added. The mixture is then esterified at 120° C. until an AN of <10 has been reached. Adipic acid and acrylic acid are added successively and esterified until an AN of <10 has been reached. The catalyst is neutralized using aqueous alkali, and residues of volatile constituents are removed under reduced pressure. The batch is filtered and compounded. The resultant product has the following properties:

| | |
|---|---|
| AN | 9.5 |
| SN | 169 |
| DP | 77° C. |
| MV 90 | 350 mPas |

### Example 3

| | | |
|---|---|---|
| Pentaerythritol | 1.0 | mol |
| Stearic acid | 2.5 | mol |
| Methyl acrylate | 1.1 | mol |
| Sn catalyst | 0.15% | by weight |

The stearic acid is melted, pentaerythritol and catalyst are added, and the mixture is then heated and esterified at 190° C. until an AN of <10 has been reached. The batch is cooled to 120° C., and methyl acrylate is added and transesterified until methanol no longer distils off. The catalyst is precipitated, and residues of volatile constituents are removed under reduced pressure, and the batch is then filtered and compounded. The resultant product has the following properties:

| | |
|---|---|
| AN | 9.5 |
| SN | 194 |
| DP | 55° C. |
| MV 90 | 50 mPas |

### Example 4

| | | |
|---|---|---|
| Pentaerythritol | 1.0 | mol |
| Stearic acid | 0.5 | mol |
| Montanic acid | 2.0 | mol |
| Acrylic acid | 1.1 | mol |
| Sn catalyst | 0.15% | by weight |
| Methanesulfonic acid, 70% | 0.1% | by weight, based on the batch |

Montanic acid and technical-grade stearic acid are melted, and pentaerythritol and catalyst are added. The mixture is then heated and esterified at 190° C. until an AN of <10 has been reached. The batch is cooled to 120° C., methanesulfonic acid is added, acrylic acid is metered in and esterified until an AN of <10 has been reached. The catalyst is neutralized, volatile constituents are distilled off under reduced pressure, and the batch is filtered and compounded. The resultant product has the following properties:

| | |
|---|---|
| AN | 9.5 |
| SN | 175 |
| DP | 68° C. |
| MV 90 | 150 mPas |

### Example 5

| | | |
|---|---|---|
| Pentaerythritol | 1.0 | mol |
| Montanic acid | 2.5 | mol |
| Acrylic acid | 1.1 | mol |
| Methanesulfonic acid, 70% | 0.15% | by weight |

The montanic acid is melted, and trimethylolpropane and methanesulfonic acid are added. The mixture is then esterified at 120° C. until an AN of <10 has been reached. Acrylic acid is then added and esterified until an AN of <10 has been reached. The catalyst is neutralized using aqueous alkali, and residues of volatile constituents are removed under reduced pressure. The batch is then filtered and compounded. The resultant product has the following properties:

| | |
|---|---|
| AN | 13 |
| SN | 172 |
| DP | 76° C. |
| MV 100 | 223 mPas |

In all compounds in Examples 1-5, the incorporation of the acrylic acid can be confirmed by 13-C-NMR.

### Use Examples

### Example 6

| UV-curing paint: | |
|---|---|
| Laromer ® PO 84 F (BASF) | 92.7% |
| Irgacure ® 500 (Ciba) | 3.0% |
| Tego Glide ® 435 (Tego-Chemie) | 0.3% |
| Wax from Example 5 | 4.0% |

Laromer PO 84 F: unsaturated acrylate resin and reactive thinner for the radiation curing of UV- and electron beam-curable paints and printing inks on various substrates.

Irgacure 500: eutectic mixture of Irgacure 184 and benzophenone

Tego Glide 435: additive recommended for aqueous and UV systems: 0.5-1% for better substrate wetting, flow and sliding properties

### Example 7

Coating of paper and wood surfaces

| | |
|---|---|
| Paper | 135 g/m² |
| Application rate | 20 g/m² |
| Baking rate | 50 m/min |
| Power | 200 W/cm² |
| Lamp-substrate distance | 100 mm |

| | | | Residue from toluene |
|---|---|---|---|
| Coating (micronized) | Gloss | Sliding friction | extract, ppm |
| Paint without additive | 89% | 0.35 | not detectable |
| Sample from Example 5 | 88% | 0.26 | not detectable |
| PE/amide wax | 55% | 0.27 | 250 |
| Amide wax | 79% | 0.295 | 350 |
| PE/PTFE | 74% | 0.265 | 180 |
| PE wax | 72% | 0.245 | 120 |

Abbreviations used:
SN saponification number
DP dropping point
MV melt viscosity at the stated temperature
PE polyethylene wax PE130
Amide wax bisstearoyl/palmitoylethylenediamine, wax C

1. A compound of the formula
Zₖ—(Y—(W)₍ₘ₋ₚ₋₁₋ₖ₎(X)_{q})ₙ
in which
Z is the radical obtained by removing the OH groups from COOH groups of an organic carboxylic acid containing 1-4 COOH groups and whose number of carbon atoms is between 4 and 40,
Y is the radical obtained by removing (m-p) hydrogen atoms from the hydroxyl groups of an organic compound having 3 to 15 carbon atoms and containing m OH groups, where Y can be a radical alkoxylated by C₂-C₄-alkylene oxides, and of the total number m of OH groups, (m-p) OH groups have been esterified and p OH groups are in free or etherified form,
X is the monovalent radical obtained by removing the OH group from the COOH group of an organic monocarboxylic acid having 3-5 carbon atoms which contains an olefinic double bond which is in conjugation with the C—O double bond,
W is the monovalent radical obtained by removing the OH group from the COOH group of the monobasic carboxylic acid having 16-60 carbon atoms,
p is a number from 0 to 2.5 which indicates the number of free or etherified OH groups,
k is a number from 0 to 2,
q is a number from 0.5 to 1.5, which indicates the number of ester units in the unsaturated acid X,
n is an integer from 1 to 6, with the proviso that k is >0, in which case n indicates the number of recurring units in the compound according to the invention,
m is an integer from 3 to 8 and indicates the number of OH groups in the polyhydric alcohol,
with the proviso that
1≦n.q≦6,
2≦(n[m-p-1])≦15,
m-p-1>0, and
m-n-1-k>1.

2. A compound as claimed in claim 1, in which monocarboxylic acids W having 22 to 40 carbon atoms are used.

3. A compound as claimed in claim 1, in which acrylic acid, methacrylic acid or an ester thereof is used as ethylenically unsaturated acid X.

4. The use of a compound as claimed in claim 1 as copolymerizable, wax-like additive to surface coatings.
